(19) United States
(12) Patent Application Publication
(10) Pub. No.: US 2016/0317436 A1
(43) Pub. Date: Nov. 3, 2016

(54) Skin Repair Compound

(71) Applicant: Dana Canuso, Atco, NJ (US)

(72) Inventor: Dana Canuso, Atco, NJ (US)

(21) Appl. No.: 15/140,324

(22) Filed: Apr. 27, 2016

Related U.S. Application Data
(60) Provisional application No. 62/154,099, filed on Apr. 28, 2015.

(51) Int. Cl. A61K 9/00; A61K 31/4174; A61K 31/27

(52) U.S. Cl. CPC A61K 9/0014; A61K 31/27; A61K 31/4174

(57) ABSTRACT

A non-biologically-derived serum formulation is provided for the treatment of skin ailments comprising an anti-fungal agent as an active ingredient, whereby the serum is prepared such that it may be applied topically to a target area for therapy. Preferably, the anti-fungal agent is tolnaftate. In some embodiments, the quantity of anti-fungal is about 1% by weight. Other quantities are contemplated and may be effective as skin care therapy. Other anti-fungal agents are also contemplated.

### BACKGROUND

The application claims priority to provisional patent application U.S. Ser. No. 62/154,099 filed on Apr. 28, 2015, the entire contents of which is herein incorporated by reference.

### BACKGROUND

The present invention relates to therapeutic compounds for treating areas of dry, rough and/or cracked skin, particular those on vulnerable areas of a patient's foot. In many cases, the ailment is fungus-based, necessitating an anti-fungal therapy. Presently, there are anti-fungal creams available, including those using the active ingredient tolnaftate. However, those compounds have limited superficial penetration and, thus, have limited effectiveness.

### SUMMARY OF CERTAIN EMBODIMENTS

A non-biologically-derived serum formulation is provided for the treatment of skin ailments comprising an anti-fungal agent as the active ingredient, whereby the serum is prepared such that it may be applied topically to a target area for therapy. A serum in the context of the invention herein is a non-biologically-derived compound with an active ingredient where the compound is formulated so as to be easily absorbed into a patient's skin and to penetrate more deeply into the skin, and wherein the compound exhibits a non-greasy finish. Preferably, the serum comprises an anti-fungal agent, such as tolnaftate or clotrimazole, by way of example. In some embodiments, the quantity of anti-fungal is about 1% by weight. Other quantities are contemplated and may be effective as skin care therapy. Other anti-fungal agents are also contemplated.

### DETAILED DESCRIPTION OF CERTAIN EMBODIMENTS

In some embodiments of the present invention, a serum is provided that comprises an active anti-fungal ingredient. With a serum of the type contemplated by the present invention, the size of the active ingredient molecule is smaller and, thereby, able to penetrate a patient's dermis more readily, and therefore, more effectively. Prior art creams are different than the present inventive serums because the active ingredient in creams have a molecular size that is too large to penetrate deeply into the dermis. In a serum, the molecular size is sufficiently small to penetrate to a more therapeutic depth. It is contemplated that embodiments of the present invention comprise a serum using an anti-fungal active ingredient such as, by way of example only, tolnaftate or clotrimazole.

In some forms, the compound is prepared for topical application in a concentrated form permitting the user to apply it to the desired area multiple times a day. What makes embodiments of the present invention different than other anti-fungal products is that is they are deliverable into the skin as a concentrated serum.

In some applications, a user can use their hands to massage the compound onto each foot, where it can be quickly absorbed so as to be dry to the touch within seconds. There is no sticky residue and the serum does not rub off. With some embodiments, the user does not need to wash their hands after applying the product.

Tests of at least one embodiment of the present invention have shown that, by applying it twice a day to a patient's foot, the user will see a softer and smoother appearance of the skin on their feet in as little as two days. Once healthy smooth skin has been obtained, the fungal infection is typically deemed to have been treated. This product can be used twice daily for treatment of dry skin and then once daily for maintenance of smooth feet.

In particular embodiments, the compound comprises the following ingredient: deionized water, propylene glycol, PEG-12 dimethicone, hydroxyethylacrylate/sodium acryloyldimethyl taurate copolymer, squalane, polysorbate 60, talc, glycerin, menthol (and) menthyl lactate, cyclopentasiloxane, dimethiconol, phenyl trimethicone, tolnaftate, olea europaea (olive) oil, phenoxyethanol, benzoic acid, dehydroacetic acid, ethoxydiglycol, polysorbate 80, cetyl acetate, and acetylated lanolin alcohol, where the percentage of tolnaftate is 1% by weight, for example. In other embodiments, more or less tolnaftate may be used. In still other embodiments, anti-fungal agents other than tolnaftate may be used, including for example clotrimazole. Also, with the present invention, moisturizing agents may be added to the compound to supplement and/or enhance hydration of the target area being treated.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A non-biologically-derived serum formulation for the treatment of skin ailments comprising an anti-fungal agent as an active ingredient, whereby the serum formulation is prepared such that it may be applied topically to a target area for therapy.

2. The serum of claim 1, where in the anti-fungal agent comprises tolnaftate.

3. The serum of claim 1, where in the anti-fungal agent comprises clotrimazole.

4. The serum of claim 1, where in the quantity of anti-fungal is 1% by weight.
